(19) United States
(12) Patent Application Publication
(10) Pub. No.: US 2023/0025603 A1
(43) Pub. Date: Jan. 26, 2023

(54) SUN CARE COMPOSITION

(71) Applicant: L'OREAL, PARIS (FR)

(72) Inventors: Tomomi HAMAZAKI, Kawasaki-shi, Kanagawa (JP); Toru KOIKE, Kanagawa (JP)

(21) Appl. No.: 17/757,174

(22) PCT Filed: Dec. 11, 2020
(86) PCT No.: PCT/JP2020/047307

§ 371 Date: Jun. 10, 2022

(30) Foreign Application Priority Data
Dec. 17, 2019 (JP) 2019-227503

(51) Int. Cl. A61K 8/02; A61K 8/25; A61Q 17/04

(52) U.S. Cl. CPC A61K 8/0279; A61K 8/25; A61Q 17/04; A61K 2800/412

(57) ABSTRACT

The present invention relates to a non-pulverulent cosmetic composition, comprising: (a) at least one lipophilic organic UV filter, and (b) at least one hollow silica sphere particle. The composition according to the present invention can exhibit a good C UV filtering effect.

### TECHNICAL FIELD

The present invention relates to a sun care composition, in particular a sun care non-pulverulent cosmetic composition, for a keratinous substance such as skin.

### BACKGROUND ART

Hollow particles are widely used as additives or fillers in many technical fields including cosmetics.

For example, JP-A-2009-57315 discloses a cosmetic comprising hollow silicone-based fine particles (A). Also, JP-A-2010-53200 discloses a light reflecting material which is composed of hollow particles constituted of a silicon oxide-based porous shell having a plurality of micropores and reflects on average 90% or more of light in the wavelength range of ultraviolet light to infrared light.

For sun care products, good UV protecting effects and good usability are among the key functions. Therefore, there is a still need for sun care compositions which exhibit good UV protecting effects.

### DISCLOSURE OF INVENTION

An objective of the present invention is to provide a sun care composition having a good UV protecting property.

The above objective of the present invention can be achieved by a non-pulverulent cosmetic composition, comprising:
(a) at least one lipophilic organic UV filter, and
(b) at least one hollow silica sphere particle.

The (b) hollow silica sphere particle may be non-porous.

The (b) hollow silica sphere particle may have an average primary particle size of 0.5 μm or more, preferably 1 μm or more, more preferably 1.5 μm or more, and in particular 2 μm or more, and of 100 μm or less, preferably 50 μm or less, more preferably 10 μm or less, and in particular 6 μm or less.

The (b) hollow silica sphere particle may have a specific surface area determined by BET method of 50 m²/g or less, preferably 30 m²/g or less, more preferably 10 m²/g or less, and even more preferably 5 m²/g or less, and of 0.1 m²/g or more, preferably 0.5 m²/g or more, more preferably 1 m²/g or more, and even more preferably 2 m²/g or more.

The (b) hollow silica sphere particle may have a void ratio of 70% by volume or less, preferably 60% by volume or less, and more preferably 50% by volume or less, and even more preferably 40% by volume or less, and of 10% by volume or more, preferably 20% by volume or more, more preferably 25% by volume or more, and even more preferably 30% by volume or more.

The (b) hollow silica sphere particle may have an oil absorption capacity of 200 mL/100g or less, preferably 150 mL/100g or less, more preferably 100 mL/100g or less, and even more preferably 70 mL/100g or less, and of 10 mL/100g or more, preferably 20 mL/100g or more, more preferably 30 mL/100g or more, and even more preferably 40 mL/100g or more.

The (b) hollow silica sphere particle may have a specific gravity of 1.1 g/cm³ or more, preferably 1.2 g/cm³ or more, and more preferably 1.2 g/cm³ or more, and 1.7 g/cm³ or less, preferably 1.6 g/cm³ or less, and more preferably 1.5 g/cm³ or less.

The (a) lipophilic organic UV filter may comprise a combination of at least one lipophilic organic UV-A filter and at least one lipophilic organic UVB filter.

The (a) lipophilic organic UV filter may be selected from minobenzophenone compounds, such as diethylamino hydroxybenzoyl hexyl benzoate (DHHB), dibenzoylmethane compounds, such as butyl methoxydibenzoylmethane, triazine compounds, such as ethylhexyl triazone, salicylic compounds, such as homosalate, β,β-diphenylacrylate compounds, such as octocrylene, and benzotriazole compounds, such as drometrizole trisiloxane, and mixtures thereof.

The composition according to the present invention may be in the form of a W/O emulsion or a solution.

The amount of the (a) lipophilic organic UV filter may be 1% by weight or more, preferably 5% by weight or more, more preferably 10% by weight or more, and even more preferably 15% by weight or more, and may be 50% by weight or less, preferably 40% by weight or less, more preferably 30% by weight or less, and even more preferably 25% by weight or less, relative to the total weight of the composition.

The amount of the (b) hollow silica sphere particle may be 0.5% by weight or more, preferably 1% by weight or more, more preferably 2% by weight or more, and even more preferably 3% by weight or more, and may be 40% by weight or less, preferably 30% by weight or less, more preferably 20% by weight or less, and even more preferably 15% by weight or less, relative to the total weight of the composition.

The composition according to the present invention may include inorganic fillers other than hollow silica particles in an amount of 10% by weight or less, preferably 5% by weight or less, more preferably 3% by weight or less, and even more preferably 1% by weight or less.

The present invention also relates to a cosmetic process for a keratin substrate, such as skin, comprising applying to the keratin substrate the composition according to the present invention.

The present invention also relates to a use of (b) at least one hollow silica sphere particle to enhance UV absorbance of a non-pulverulent composition comprising (a) at least one lipophilic organic UV filter.

### BRIEF DESCRIPTION OF DRAWINGS

FIG. 1 shows a graph of UV absorbance of each of the compositions according to

### EXAMPLE 1 and COMPARATIVE EXAMPLES 1 and 2

### BEST MODE FOR CARRYING OUT THE INVENTION

UV filters are widely used to impart a UV protecting property to sun care composition. However, the use of UV filters may cause negative impacts, such as oily and greasy feelings.

After diligent research, the inventors have surprisingly discovered that hollow silica sphere particles can enhance a UV protecting property of non-pulverulent compositions comprising at least one lipophilic organic UV filter, and thus completed the present invention.

Thus, the present invention relates to a non-pulverulent cosmetic composition, comprising:
(a) at least one lipophilic organic UV filter, and
(b) at least one hollow silica sphere particle.
   Hereinafter, the particle, composition, process, and use according to the present invention will be explained in a more detailed manner.

### [Composition]

The composition according to the present invention is a non-pulverulent cosmetic composition. The term “non-pulverulent composition” herein means any composition that is not in the form of a loose or compact powder. The non-pulverulent composition may take various forms, such as, a solution, a gel, a lotion, a serum, a suspension, a dispersion, a fluid, a milk, a paste, a cream, an emulsion (O/W or W/O form), or the like. It is preferable that the composition according to the present invention be in the form of a W/O emulsion.

The composition according to the present invention may be intended for use as a cosmetic topical composition. Thus, the composition according to the present invention may be intended for application onto a keratinous substance. Keratinous substance here means a material containing keratin as a main constituent element, and examples thereof include the skin, scalp, nails, lips, hair, and the like. In particular, the composition according to the present invention may be a skin sun care cosmetic composition for protecting skin from UV rays.

The pH of the composition according to the present invention is not particularly limited. The pH of the composition may be from 2.0 to 12.0, preferably 3.0 to 11.0, and more preferably 4.0 to 10.0.

The viscosity of the composition according to the present invention is not particularly limited. Preferably, the viscosity of the composition according to the present invention ranges from 1 to 1,000,000 mm²/s, more preferably from 2 mm²/s to 500,000 mm²/s, and even more preferably from 5 mm²/s to 100,000 mm²/s at 25° C. The viscosity of the composition according to the present invention can be measured using a Poiseuille rheometer, at a temperature of 25° C., according to standard ASTM-D445-97.

The inventors have surprisingly discovered that hollow silica sphere particles can increase a UV absorbance of non-pulverulent compositions comprising at least one lipophilic organic UV filter. Therefore, the composition according to the present invention can achieve a sufficient UV protecting property even if it does not include a large amount of UV filters, such as lipophilic organic UV filters, which may cause negative impacts on cosmetic compositions in terms of, for example, an oily or greasy texture. Accordingly, the composition according to the present invention can exhibit a good UV protecting effects as well as good texture in consideration of a reduced amount of UV filters, in particular lipophilic organic UV filters, in sun care non-pulverulent compositions.

The composition according to the present invention includes (a) at least one lipophilic organic UV filter, and (b) at least one hollow silica sphere particle. The ingredients in the composition will be described in a detailed manner below

### (Lipophilic Organic UV Filter)

The composition according to the present invention comprises (a) at least one lipophilic organic UV filter. Two or more lipophilic organic UV filters may be used in combination. Thus, a single type of lipophilic organic UV filter or a combination of different types of lipophilic organic UV filters may be used.

The term “UV” here comprises the UV-B region (260-320 nm in wavelength), the UV-A region (320-400 nm in wavelength), and the high energy visible light region (400-450 nm in wavelength). Therefore, a UV filter means any material which has filtering effects in the wavelength of UV rays, in particular the UV-A, UV-B, and high energy visible light regions.

The UV filter(s) used for the present invention may be active in the UV-A and/or UV-B region, preferably in both of the UV-A and UV-B regions alone or in combination. Therefore, the UV filter(s) used in the present invention include(s) a UV-A filter capable of absorbing UV radiation from 320 to 400 nm, a UV-B filter capable of absorbing UV radiation from 280 to 320 nm, and a UV-A and UV-B filter capable of absorbing UV radiation from 280 to 400 nm.

The term “lipophilic UV filter” here means UV filters which are soluble in oils at a concentration of at least 1% by weight relative to the total weight of the oils at room temperature (25° C.) and atmosphere pressure (10⁵ Pa).

The lipophilic organic UV filter may be solid or liquid. The terms “solid” and “liquid” mean solid and liquid, at room temperature (25° C.) and atmosphere pressure (105 Pa).

The lipophilic organic UV-A filters used in the present invention may include, but are not limited to, aminobenzophenone compounds, dibenzoylmethane compounds, anthranilic acid compounds, and 4,4-diarylbutadiene compounds.

As the aminobenzophenone compounds, mention may be made of n-hexyl 2-(4-diethlamino-2-hydroxybenzoyDbenzoate, the alternative name of which is diethylamino hydroxybenzoyl hexyl benzoate (DHHB), sold under the trade name “Uvinul A+” from BASF.

As the dibenzoylmethane compounds, mention may be made of 4-isopropyldibenzoylmethane, sold under the name of “Eusolex 8020” from Merck, 1-(4-methoxy-l-benzofuran-5-yl)-3-phenylpropane-1,3-dione, sold under the name of “Pongamol” from Quest, 1-(4-(tert-butyl)phenyl)-3-(2-hydroxyphenyl)propane-1,3-dione, and butyl methoxydibenzoylmethane, sold under the trade name “Parsol 1789” from Hoffmann-La Roche.

As the anthranilic acid compounds, mention may be made of menthyl anthranilate marketed under the name “NEO HELIPAN MA” by Symrise.

As the 4,4-diarylbutadiene compounds, mention may be made of 1,1 -dicarboxy (2,2′-dimethylpropyl)-4,4-diphenylbutadiene and diphenyl butadiene malonates and malononitriles.

The lipophilic organic UV-B filters used in the present invention may include, but are not limited to, triazine compounds, para-aminobenzoic acid compounds, salicylic compounds, cinnamate compounds, β,β-diphenylacrylate compounds, benzylidenecamphor compounds, phenylbenzimidazole compounds, imidazoline compounds, benzalmalonate compounds, and mecocyanine compounds.

As the triazine compounds, mention may be made of ethylhexyl triazone, marketed under the name “UVINUL T-150” by BASF, diethylhexyl butamido triazone, marketed under the name “UVASORB HEB” by SIGMA 2V, 2,4,6-tris(dineopentyl 4′-aminobenzalmalonate)-s-triazine, 2,4,6-tris(diisobutyl 4′-aminobenzalmalonate)-s-triazine, 2,4-bis(dineopentyl 4′-aminobenzalmalonate)-6-(n-butyl 4′-aminobenzoate)-s-triazine, and 2,4-bis(n-butyl 4′-aminobenzoate)-6-(aminopropyltrisiloxane)-s-triazine.

As the para-aminobenzoic acid derivatives, mention may be made of para-aminobenzoates (PABA), for example, ethyl PABA (para-aminobenzoate), ethyl dihydroxypropyl PABA, and ethylhexyl dimethyl PABA, marketed under the name “ESCALOL 5972 from ISP.

As the salicylic compounds, mention may be made of homosalate, marketed under the name “Eusolex HMS” by Rona/EM industries, and ethylhexyl salicylate, marketed under the name “NEO HELIOPAN OS” by Symrise.

As the cinnamate compounds, mention may be made of ethylhexyl methoxycinnamate, marketed under the name “PARSOL CX” by DSM NUTRITIONAL PRODUCTS, isopropyl ethoxy cinnamate, isoamyl methoxy cinnamate, marketed under the name “NEO HELIOPAN E 1000” by Symrise, diisopropyl methylcinnamate, cinoxate, and glyceryl ethylhexanoate dimethoxycinnamate.

As the β,β-diphenylacrylate compounds, mention may be made of octocrylene, marketed under the name “UVINUL N539” by BASF, and etocrylene, marketed under the name “UVINUL N35” by BASF.

As the benzylidenecamphor compounds, mention may be made of 3-benzylidene camphor, marketed under the name “MEXORYL SD” from CHIMEX, methylbenzylidene camphor, marketed under the name “EUSOLEX 6300” by MERCK, polyacrylamidomethyl benzylidene Camphor, marketed under the name “MEXORYL SW” by CHIMEX, and terephthalylidene dicamphor sulfonic acid, marketed under the name “Mexoryl SX” by Chimex.

As the phenylbenzimidazole compounds, mention may be made of phenylbenzimidazole sulfonic acid, marketed under the name “Eusolex 232” by Merck, and disodium phenyl dibenzimidazole tetrasulfonate, marketed under the name “Neo Heliopan AP” by Haarmann and Reimer.

As the imidazoline compounds, mention may be made of ethylhexyl dimethoxybenzylidene dioxoimidazoline propionate.

As the benzalmalonate compounds, mention may be made of polyorganosiloxane containing a benzalmalonate moiety, for example, Polysilicone-15, marketed under the name “Parsol SLX” by DSM NUTRITIONAL PRODUCTS, and di-neopentyl 4′-methoxybenzalmalonate.

The lipophilic organic UV filters of the present invention may comprise lipophilic organic UV-A and UV-B filters. The following are non-limited examples of the lipophilic organic UV-A and UV-B filters:
Benzophenone compounds, such as benzophenone-1 marketed underthe name “UVINUL 400” by BASF, benzophenone-2 marketed under the name “UVINUL 500” by BASF, benzophenone-3 or oxybenzone marketed under the name “UVINUL M40” by BASF, benzophenone-6 marketed under the name “Helisorb 11” by NOrquay, benzophenone-8 marketed under the name “Spectra-Sorb UV-24” by American Cyanamid, benzophenone-10, benzophenone-11, and benzophenone-12;
benzotriazole compounds such as drometrizole trisiloxane marketed under the name “Silatrizole” by Rhodia Chimie, bumetrizole marketed under the name “TINOGUARTD AS” by CIBA-GEIGY, and, phenylbenzotriazole derivatives: 2-(211-benzotriazole-2-yl)-6-dodecyl-4-methylpheno, branched and linear;
bis-resorcinyl triazine compounds, such as bis-ethylhexyloxyphenol methoxyphenyl triazine marketed under the name “TINOSORB S” by CIBA-GEIGY; and
benzoxazole compounds, such as 2,4-bis[5-(1-dimethylpropyl) benzoxazol-2-yl(4-phenypimino]-6-(2-ethylhexyl)imino-1,3,5-triazine marketed under the name “Uvasorb K2A” by Sigma 3V.

Preferably, the lipophilic organic UV filter may be selected from aminobenzophenone compounds, such as diethylamino hydroxybenzoyl hexyl benzoate (DHHB), dibenzoylmethane compounds, such as butyl methoxydibenzoylmethane, triazine compounds, such as ethylhexyl triazone, salicylic compounds, such as homosalate, β,β-diphenylacrylate compounds, such as octocrylene, and benzotriazole compounds, such as drometrizole trisiloxane, and mixtures thereof.

In one preferred embodiment of the present invention, the lipophilic organic UV filter of the present invention comprise a combination of at least one lipophilic organic UV-A filter and at least one lipophilic organic UV-B filter.

Therefore, in another preferred embodiment of the present invention, the lipophilic organic UV filter comprises at least one lipophilic organic UV-A filter selected from aminobenzophenone compounds and dibenzoylmethane compounds, and at least one lipophilic organic UV-B filter selected from triazine compounds, salicylic compounds, β,β-diphenylacrylate compounds, and benzotriazole compounds.

The amount of the lipophilic organic UV filter(s) in the composition according to the present invention may be 1% by weight or more, preferably 5% by weight or more, more preferably 10% by weight or more, and even more preferably 15% by weight or more, relative to the total weight of the composition. The amount of the lipophilic organic UV filter(s) in the composition may be 50% by weight or less, preferably 40% by weight or less, more preferably 30% by weight or less, and even more preferably 25% by weight or less, relative to the total weight of the composition.

In one particular embodiment of the present invention, the amount of the lipophilic organic UV-A filter(s) in the composition is 0.5% by weight or more, preferably 1% by weight or more, and more preferably 2% by weight or more, and is 15% by weight or less, preferably 10% by weight or less, and more preferably 7% by weight or less, relative to the total weight of the composition.

In another particular embodiment of the present invention, the amount of the lipophilic organic UV-B filter(s) in the composition is 1% by weight or more, preferably 5% by weight or more, and more preferably 10% by weight or more, and is 30% by weight or less, preferably 20% by weight or less, and more preferably 16% by weight or less, relative to the total weight of the composition.

### (Hollow Silica Sphere Particle)

The composition according to the present invention comprises (b) at least one hollow silica sphere particle. Two or more hollow silica sphere particles may be used in combination. Thus, a single type of hollow silica sphere particle or a combination of different types of hollow silica sphere particles may be used.

The particle used in the composition according to the present invention is a silica-based particle. The silica-based particle has an outer shell and a cavity inside the outer shell to form a hollow structure. In addition, the shape of particle of the present invention is a spherical shape.

The outer shell of the hollow silica sphere particle can be porous or non-porous. Preferably, the outer shell of the hollow silica sphere particle is non-porous.

The size of the hollow silica sphere particle of the present invention is not particularly limited. In general, the hollow silica sphere particle has an average primary particle size of from 0.1 μm to 200 μm. Preferably, the hollow silica sphere particle has an average primary particle size of 0.5 μm or more, more preferably 1 μm or more, even more preferably 1.5 μm or more, and in particular 2 μm or more. Preferably, the hollow silica sphere particle has an average primary particle size of 100 μm or less, more preferably 50 μm or less, even more preferably 10 μm or less, and in particular 6 μm or less.

The term “average primary particle size” used herein represents a number-average size mean diameter which is given by the statistical particle size distribution to half of the population, referred to as D50. For example, the number-average size mean diameter can be measured by a laser diffraction particle size distribution analyzer, such as Mastersizer 2000 by Malvem Corp.

The hollow silica sphere particle of the present invention may have a specific surface area determined by BET method of 50 m²/g or less, preferably 30 m²/g or less, more preferably 10 m²/g or less, and even more preferably 5 m²/g or less. The hollow silica sphere particle may have a specific surface area determined by BET method of 0.1 m²/g or more, preferably 0.5 m²/g or more, more preferably 1 m²/g or more, and even more preferably 2 m²/g or more.

In the present invention, the “specific surface area determined by BET method” can mean a value determined by: drying a sample for measurement at 200° C. for no less than three hours under a reduced pressure of no more than 1 kPa; thereafter measuring an adsorption isotherm of only nitrogen adsorption at liquid nitrogen temperature; and analyzing the adsorption isotherm by the BET method. The pressure range used for the analysis is a relative pressure of 0.1 to 0.25.

The hollow silica sphere particle of the present invention may have a void ratio according to the cavity inside the outer shell of 70% by volume or less, preferably 60% by volume or less, and more preferably 50% by volume or less, and even more preferably 40% by volume or less. The hollow silica sphere particle may have a void ratio of 10% by volume or more, preferably 20% by volume or more, more preferably 25% by volume or more, and even more preferably 30% by volume or more.

The void ratio herein can be measured by, for example, an image analysis. Specifically, the void ratio can be determined by taking a TEM photograph of the particles, measuring the particle diameter of the particles for, for example, 50 particles, determining the average particle diameter, breaking the particles by half, measuring the diameter of the cavity for each fractured pieces to determine the average diameter of the cavity, and then calculating the void ratio.

The hollow silica sphere particle of the present invention may have an oil absorption capacity of 200 mL/100 g or less, preferably 150 mL/100 g or less, more preferably 100 mL/100 g or less, and even more preferably 70 mL/100 g or less. The hollow silica sphere particle of the present invention may have an oil absorption capacity of 10 mL/100 g or more, preferably 20 mL/100 g or more, more preferably 30 mL/100 g or more, and even more preferably 40 mL/100 g or more. The oil absorption capacity herein can be measured with a pigment test method in accordance with JIS-K5101 using boiled linseed oil.

The hollow silica sphere particle of the present invention may have an oil absorption capacity of 200 mL/100 g or less, preferably 150 mL/100 g or less, more preferably 100 mL/100 g or less, and even more preferably 70 mL/100 g or less. The hollow silica sphere particle of the present invention may have an oil absorption capacity of 10 mL/100 g or more, preferably 20 mL/100 g or more, more preferably 30 mL/100 g or more, and even more preferably 40 mL/100 g or more. The oil absorption capacity herein can be measured with a pigment test method in accordance with JIS-K5101 using boiled linseed oil.

The specific gravity of the hollow silica sphere particle of the present invention is not particularly limited. The specific gravity of the hollow silica sphere particle may be generally 1.1 g/cm³ or more, preferably 1.2 g/cm³ or more, and more preferably 1.3 g/cm³ or more, and 1.7 g/cm³ or less, preferably 1.6 g/cm³ or less, and more preferably 1.5 g/cm³ or less.

The hollow silica sphere particle of the present invention may have a refractive index of 1.5 or less, preferably 1.4 or less, and more preferably 1.3 or less. The lower limit of the refractive index of the hollow silica sphere particle is not limited, but in general 1.0 or more, preferably 1.1 or more, and more preferably 1.2 or more.

In one embodiment of the present invention, the cavity inside the outer shell of the hollow silica sphere particle of the present invention may be spherical and may have a diameter size of 0.5 μm or more, preferably 1 μm or more, even more preferably 1.5 μm or more, and of 50 μm or less, preferably 10 μm or less, more preferably6 μm or less. The diameter size of the spherical cavity in the hollow silica sphere particle can be measured by, for example, an image analysis using TEM as mentioned above. Specifically, it can be measured by breaking the particles by half, taking a TEM photograph of the fractured pieces of the particles, and measuring the diameter of the cavity for each fractured pieces.

The amount of the hollow silica sphere particle in the composition according to the present invention may be 0.5% by weight or more, preferably 1% by weight or more, more preferably 2% by weight or more, and even more preferably 3% by weight or more, relative to the total weight of the composition. The amount of the hollow silica sphere particle in the composition may be 40% by weight or less, preferably 30% by weight or less, more preferably 20% by weight or less, and even more preferably 15% by weight or less, relative to the total weight of the composition.

### (Other Ingredients)

### Oil

The composition according to the present invention may comprise at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

As examples of plant oils, mention may be made of, for example, linseed oil, camellia oil, macadamia nut oil, corn oil, olive oil, avocado oil, sasanqua oil, castor oil, safflower oil, jojoba oil, sunflower oil, almond oil, rapeseed oil, sesame oil, soybean oil, peanut oil, and mixtures thereof. As examples of animal oils, mention may be made of, for example, squalene and squalane. As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched. Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

As examples of ether oils, mention may be made of, for example, ether oils with a short hydrocarbon chain or chains, such as dicaprylyl ether.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof. Preferably, the silicone oil is chosen from liquid polydialkylsiloxanes, especially liquid polydimethylsiloxanes (PDMS, dimethicone) and liquid polyorganosiloxanes comprising at least one aryl group. These silicone oils may also be organomodified. The organomodified silicones that can be used in accordance with the present invention are silicone oils as defined above and comprise in their structure one or more organofunctional groups attached via a hydrocarbon-based group.

Hydrocarbon oils may be chosen from:
linear or branched, optionally cyclic, C₆-C₁₆ lower alkanes. Examples that may be mentioned include hexane, undecane, dodecane, tridecane, and isoparafrms, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group. As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof. It is preferable that the fatty alcohol be a saturated fatty alcohol. Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols. As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

The amount of the oil(s) in the composition according to the present invention may range from 5 to 60% by weight, preferably from 10 to 50% by weight, and more preferably from 15 to 40% by weight, relative to the total weight of the composition.

### Cosmetically Acceptable Hydrophilic Organic Solvent

The composition according to the present invention may comprise at least one cosmetically acceptable hydrophilic organic solvent. The cosmetically acceptable hydrophilic organic solvent(s) may include, for example, substantially linear or branched lower mono-alcohols having from 1 to 8 carbon atoms, such as ethanol, propanol, butanol, isopropanol, and isobutanol; aromatic alcohols, such as benzyl alcohol and phenylethyl alcohol; polyols or polyol ethers, such as propylene glycol, dipropylene glycol, isoprene glycol, butylene glycol, glycerine, propanediol, caprylyl glycol, sorbitol, ethylene glycol monomethyl, monoethyl and monobutyl ethers, propylene glycol ethers, such as propylene glycol monomethylether, diethylene glycol alkyl ethers, such as diethylene glycol monoethylether or monobutylether; polyethylene glycols, such as PEG-4, PEG-6, and PEG-8, and their derivatives, and a combination thereof.

The amount of the cosmetically acceptable hydrophilic organic solvent(s) in the composition according to the present invention may range from 1 to 25% by weight, preferably from 2 to 20% by weight, and more preferably from 3 to 15% by weight, relative to the total weight of the composition.

### Surfactant

The composition according to the present invention may comprise at least one surfactant chosen from amphoteric, anionic, cationic, or nonionic surfactants, used alone or as a mixture.

Preferably, the composition comprises at least one nonionic surfactant.

Examples of nonionic surfactants usable in the compositions of the invention may include polyethoxylated fatty alcohols or polyglycerolated fatty alcohols, such as the adducts of ethylene xxide with lauryl alcohol, especially those containing from 9 to 50 oxyethylene units (Laureth-9 to Laureth-50 as the INCI names), in particular Laureth-9; esters of polyols and of a fatty acid possessing a saturated or unsaturated chain comprising, for example, from 8 to 24 carbon atoms, and their oxyalkylenated derivatives, that is to say comprising oxyethylene and/or oxypropylene units, such as esters of glycerol and of a C₈-C₂₄ fatty acid, and their oxyalkylenated derivatives, in particular polyoxyethylenated glyceryl stearate (mono-, di- and/or tristearate), for example PEG-20 glyceryl triisostearate; esters of sugar and of a C₈-C₂₄ fatty acid and their oxyalkylenated derivatives, such as polyethoxylated sorbitol esters of C₈-C₂₄ fatty acids, in particular Polysorbate 80, such as the product marketed under the name “TWEEN 80” by Croda; ethers of a sugar and of C₈-C₂₄ fatty alcohols, such as caprylyl/capryl glucoside; polyoxyethylene alkyl ethers; polyoxyethylene oxypropylene alkyl ethers; fatty acid alkanol amides; alkyl amine oxides; alkyl polyglycosides and silicone surfactants, such as polydimethylsiloxane containing oxyethylene groups and/or oxypropylene groups, for example, PEG-10 dimethicone, bis-PEG/PPG-14/14 dimethicone, bis-PEG/PPG-20/20 dimethicone, and PEG/PPG-20/6 dimethicone; and polyglyceryl fatty acid ester such as polyglyceryl-4 caprate, polyglyceryl-6 dicaprate, polyglyceryl-6 dicaprate, polyglyceryl-6 dioleate, polyglyceryl-6 caprylate, polyglyceryl-2 oleate, and polyglyceryl-6 polyricinoleate; and mixtures thereof.

In addition, mention can be made of alkylpolyglycosides as nonionic surfactants, represented by the following general formula (1):

R-O-(G)ₓ(1)

in which R represents a branched and/or unsaturated alkyl radical comprising from 14 to 24 carbon atoms, G represents a reduced sugar comprising 5 or 6 carbon atoms and x denotes a value ranging from 1 to 10 and preferably from 1 to 4, and G in particular denotes glucose, fructose or galactose. Mention may be made, as alkyl polyglycosides of this type, of alkyl polyglucosides (G=glucose in the formula (I)) and in particular the compounds of formula (I) in which R more particularly represents an oleyl radical (unsaturated C₁₈ radical) or isostearyl (saturated C₁₈ radical), G denotes glucose and x is a value ranging from 1 to 2, in particular isostearyl glucoside, oleyl glucoside and their mixtures. This alkyl polyglucoside can be used as a mixture with a coemulsifier, more especially with a fatty alcohol and in particular a fatty alcohol having the same fatty chain as that of the alkyl polyglucoside, that is to say comprising from 14 to 24 carbon atoms and having a branched and/or unsaturated chain, for example isostearyl alcohol when the alkyl polyglucoside is isostearyl glucoside and oleyl alcohol when the alkyl polyglucoside is oleyl glucoside. Use may be made, for example, of the mixture of isostearyl glucoside and isostearyl alcohol, sold under the name Montanov WO 18 by Seppic, and also the mixture of octyldodecanol and octyldodecyl xyloside sold under the name Fludanov 20X by Seppic:

The amount of the surfactant(s) in the composition may be from 0.1 to 15% by weight, preferably from 0.5 to 10% by weight, and more preferably from 1 to 5% by weight, relative to the total weight of the composition.

### Thickener

The composition according to the present invention may comprise at least one thickener. Two or more thickeners can be combined. The thicker may be hydrophilic or lipophilic, and preferably lipophilic.

The lipophilic thickener may be in the form of polymer or particles.

The lipophilic polymer thickener may be chosen from carboxyvinyl polymers such as the Carbopol products (carbomers) and the Pemulen products (acrylate/C10-C30-alkyl acrylate copolymer) or polymers having the INCI name “Poly C10-30 Alkyl Acrylate”, such as the Intelimer® products from Air Products, such as the product Intelimer® IPA 13-1, which is a polystearyl acrylate, or the product 30 Intelimer® IPA 13-6 which is a behenyl polymer.

The lipophilic thickener according to the present invention may be chosen from:
organomodified clays, which are clays treated with compounds chosen especially from quaternary amines and tertiary amines. Organomodified clays that may be mentioned include organomodified bentonites, such as the product sold under the name Bentone 34 by the company Rheox, and organomodified hectorites such as the products sold under the names Bentone 27 and Bentone 38 by the company Rheox. Mention may be made especially of modified clays such as modified magnesium silicate (Bentone gel® VS38 from Rheox), modified hectorites such as hectorite modified with a C10 to C22 fatty acid ammonium chloride, for instance hectorite modified with distearyldimethylammonium chloride (disteardimonium hectorite) such as the product sold under the name Bentone 38VCG by the company Elementis or the product sold under the name Bentone 38 CE by the company Rheox, or the product sold under the name Bentone Gel® V5 5V by the company Elementis, or the product sold under the name Bentone gel® ISD V by the company Elementis;
hydrophobic fumed silicas, which may be obtained by modification of the surface of the silica via a chemical reaction that generates a reduction in the number of silanol groups, these groups possibly being substituted especially with hydrophobic groups. The hydrophobic groups can be trimethylsiloxyl groups, which are obtained especially by treating fumed silica in the presence of hexamethyldisilazane. Silicas thus treated are known as “Silica silylate” according to the CTFA Dictionary (6th edition, 1995). They are sold, for example, under the references Aerosil R812® by the company Degussa and Cab-O-Sil TS-530® by the company Cabot. The hydrophobic groups can be dimethylsilyloxyl or polydimethylsiloxane groups, which are obtained especially by treating fumed silica in the presence of polydimethylsiloxane or dimethyldichlorosilane. Silicas thus treated are known as “Silica dimethyl silylate” according to the CTFA Dictionary (6th edition, 1995). They are sold, for example, under the references Aerosil R972® and Aerosil R974® by the company Degussa and Cab-O-Sil TS-610® and Cab-O-Sil TS-720® by the company Cabot.
hydrophobic silica aerogels, such as the products sold under the name VM-2260 (INCI name: Silica silylate) by the company Dow Corning, the particles of which have a mean size of about 1000 microns and a specific surface area per unit of mass ranging from 600 to 800 m²/g; mention may also be made of the aerogels sold by the company Cabot under the references Aerogel TLD 201, Aerogel OGD 201, Aerogel TLD 203, Enova® Aerogel MT 1 100, Enova Aerogel MT 1200;
and mixtures thereof.

The amount of the thickener(s) in the composition may be from 0.1 to 10% by weight, preferably from 0.2 to 5% by weight, and more preferably from 1 to 3% by weight, relative to the total weight of the composition.

### Inorganic UV Filter

The composition according to the present invention may comprise at least one inorganic UV filter. Two or more inorganic UV filters can be combined.

The inorganic UV filter used for the present invention may be active in the UV-A and/or UV-B region. The inorganic UV filter may be hydrophilic and/or lipophilic. The inorganic UV filter is preferably insoluble in solvents such as water and ethanol, commonly used in cosmetics.

It is preferable that the inorganic UV filter be in the form of a fine particle such that the mean (primary) particle diameter thereof ranges from 1 nm to 50 nm, preferably 5 nm to 40 nm, and more preferably 10 nm to 30 nm. The mean (primary) particle size or mean (primary) particle diameter here is an arithmetic mean diameter.

The inorganic UV filter can be selected from the group consisting of metal oxides which may or may not be coated, and mixtures thereof.

Preferably, the inorganic UV filters may be selected from pigments (mean size of the primary particles: generally from 5 nm to 50 nm, preferably from 10 nm to 50 nm) formed of metal oxides, such as, for example, pigments formed of titanium oxide (amorphous or crystalline in the rutile and/or anatase form), iron oxide, zinc oxide, zirconium oxide or cerium oxide, which are all UV photoprotective agents that are well known per se. Preferably, the inorganic UV filters may be selected from titanium oxide, zinc oxide, and more preferably titanium oxide.

The inorganic UV filter may or may not be coated. The inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes such as beeswax, (meth)acrylic polymers, organic UV filters, and (per)fluoro compounds.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of repeated main units in which the silicon atoms are connected to one another via oxygen atoms (siloxane bond), optionally substituted hydrocarbon radicals being connected directly to said silicon atoms via a carbon atom.

The silicones used for the coating(s) can preferably be selected from the group consisting of alkylsilanes, polydialkylsiloxanes, and polyalkylhydrosiloxanes. More preferably still, the silicones are selected from the group consisting of octyltrimethylsilanes, polydimethylsiloxanes, and polymethylhydrosiloxanes.

Of course, the inorganic UV filters made of metal oxides may, before their treatment with silicones, have been treated with other surfacing agents, in particular, with cerium oxide, alumina, silica, aluminum compounds, silicon compounds, or their mixtures.

The coated inorganic UV filters may be titanium oxides coated with:
silica, such as the product “Sunveil” from Ikeda;
silica and iron oxide, such as the product “Sunveil F” from Ikeda;
silica and alumina, such as the products “Microtitanium Dioxide MT 500 SA” from Tayca, “Tioveil” from Tioxide, and “Mirasun TiW 60” from Rhodia;
alumina, such as the products “Tipaque TTO-55 (B)” and “Tipaque TTO-55 (A)” from Ishihara, and “UVT 14/4” from Kemira;
alumina and aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; alumina and aluminum laurate, such as the product “Microtitanium Dioxide MT 100 S” from Tayca;
iron oxide and iron stearate, such as the product “Microtitanium Dioxide MT 100 F” from Tayca;
zinc oxide and zinc stearate, such as the product “BR351” from Tayca;
silica and alumina and treated with a silicone, such as the products “Microtitanium Dioxide MT 600 SAS”, “Microtitanium Dioxide MT 500 SAS”, and “Microtitanium Dioxide MT 100 SAS” from Tayca;
silica, alumina, and aluminum stearate and treated with a silicone, such as the product “STT-30-DS” from Titan Kogyo;
   silica and treated with a silicone, such as the product “UV-Titan X 195” from Kemira; alumina and treated with a silicone, such as the products “Tipaque TTO-55 (S)” from Ishihara or “UV Titan M 262” from Kemira;
triethanolamine, such as the product “STT-65-S” from Titan Kogyo;
stearic acid, such as the product “Tipaque TTO-55 (C)” from Ishihara; or
sodium hexametaphosphate, such as the product “Microtitanium Dioxide MT 150 W” from Tayca. stearic acid and aluminum hydroxide, such as the product “MT-100 TV” from Tayca, with a mean primary particle diameter of 15 nm;
dimethicone and stearic acid and aluminum hydroxide, such as the product “SA-TTO-S4” from Miyoshi Kasei, with a mean primary particle diameter of 15 nm;
silica, such as the product “MT-100 WP” from Tayca, with a mean primary particle diameter of 15 nm;
dimethicone and silica and aluminum hydroxide, such as the product “MT-Y02” and “MT-Y-110 M3S” from Tayca, with a mean primary particle diameter of 10 nm;
dimethicone and aluminum hydroxide, such as the product “SA-TTO-S3” from Miyoshi Kasei, with a mean primary particle diameter of 15 nm;
dimethicone and alumina, such as the product “UV TITAN M170” from Sachtleben, with a mean primary particle diameter of 15 nm;
silica and aluminum hydroxide and alginic acid, such as the product “MT-100 AQ” from Tayca, with a mean primary particle diameter of 15 nm; and
aluminum hydroxide and dimethicone and hydrogen dimethicone, such as “SAS-UT-A30” from Miyoshi Kasei.

The amount of the inorganic UV filter(s) in the composition may be from 1% by weight or more, preferably 2% by weight or more, and more preferably 3% by weight or more, and 20% by weight or less, preferably 15% by weight or less, and more preferably 10% by weight, relative to the total weight of the composition.

### Filler

The composition according to the present invention may comprise at least filler other than hollow silica particles. Two or more fillers can be combined. The filler can be inorganic or organic. The filler also can be silicone powder.

As the inorganic filler other than hollow silica particle, mention may be made of talc, mica, non-hollow (solid) silica, magnesium aluminium silicate, trimethyl siloxysilicate or silica silylate, kaolin, bentone, calcium carbonate, magnesium hydrogen carbonate, hydroxyapatite, boron nitride, fluorphlogopite, sericite, calcinated talc, calcinated mica, calcinated sericite, synthetic mica, perlite, lauroyl lysine, metal soap, bismuth oxychloride, barium sulfate, magnesium carbonate, and mixtures thereof, optionally hydrophilic- or hydrophobic-treated.

The inorganic filler herein is different from inorganic UV filter as explained above.

As the organic filler, mention may be made of acrylic polymer powders, silicone powders, wax powders, polyamide powders, urethane polymer powders, tetrafluoroethylene polymer powders, polyacrylonitrile powders, poly-β-alanine powders, polyethylene powders, polytetrafluoroethylene powders, lauroyllysine, starch, cellulose powder, tetrafluoroethylene polymer powders and mixtures thereof.

As the silicone powders, mention may be made of organopolysilsesquioxane powders, organopolysiloxane powders, and silicone resin powders.

The organopolysilsesquioxane powders are preferably polymethylsilsesquioxane powders. Materials sold by the company Momentive Performance Materials, under the tradename “TOSPEARL” and materials sold by NIKKO RICA under the name MSP-N050 and MSP-N080 are examples of such polymethylsilsesquioxane powders.

The organopolysiloxane powders may be elastomeric or non-elastomeric. It is preferable to use elastomeric organopolysiloxane powder or organopolysiloxane elastomer powder.

The elastomeric organopolysiloxane may, for example, be crosslinked and may be obtained via a crosslinking addition reaction of diorganopolysiloxane comprising at least one hydrogen linked to silicon and of diorganopolysiloxane comprising at least one ethylenically unsaturated group linked to silicon, preferably, in the presence of, for example, a platinum catalyst; or via a dehydrogenation crosslinking condensation reaction between a diorganopolysiloxane comprising at least one hydroxyl end group and a diorganopolysiloxane comprising at least one hydrogen linked to silicon, preferably, in the presence of, for example, an organotin compound; or via a crosslinking condensation reaction of a diorganopolysiloxane comprising at least one hydroxyl end group and of a hydrolysable organopolysilane; or

via thermal crosslinking of organopolysiloxane, preferably, in the presence of, for example, an organoperoxide catalyst; or

via crosslinking of organopolysiloxane by high-energy radiation such as gamma rays, ultraviolet rays or an electron beam.

Elastomer organopolysiloxane powders that may be used include those sold under the names “Dow Corning 9505 Powder” and “Dow Corning 9506 Powder” by the company Dow Coming. These powders have the INCI name: dimethicone/vinyl dimethicone crosspolymer.

The amount of the filler(s) in the composition may be 0.1% by weight or more, preferably 0.5% by weight or more, and more preferably 1% by weight or more, and 20% by weight or less, preferably 15% by weight or less, and more preferably 10% by weight or less, relative to the total weight of the composition.

In one embodiment of the present invention, the composition according to the present invention includes inorganic fillers other than hollow silica particles in an amount of 10% by weight or less, preferably 5% by weight or less, more preferably 3% by weight or less, and even more preferably 1% by weight or less. In another embodiment of the present invention, the composition according to the present invention is free of inorganic fillers other than hollow silica particles. The inorganic fillers may be metal oxide. The inorganic fillers may be a particle thickener or may not be a particle thickener as explained above.

### Water

The composition according to the present invention can comprise water.

The amount of water in the composition may be from 1% by weight or more, preferably 5% by weight or more, more preferably 10% by weight or more, and more preferably 15% by weight or more, and 70% by weight or less, preferably 60% by weight or less, and more preferably 50% by weight or less, relative to the total weight of the composition.

### Adjuvants

The compositions according to the present invention may also contain various adjuvants conventionally used in compositions for sun care products, which may be selected from a physiologically acceptable medium, cationic, anionic, non-ionic, amphoteric or zwitterionic polymers or mixtures thereof, antioxidants, such as tocopherol, neutralizing agents, such as triethanolamine, sequestering agents, such as trisodium ethylenediamine disuccinate, buffer, such as tromethamine, fragrances, emollients, dispersing agents, dyes and/or pigments, film-foiiiiing agents and/or thickeners, ceramides, preservatives, such as phenoxy ethanol, co-preservatives and opacifying agents.

The adjuvants may be present in the composition of the present invention in an amount preferably ranging from 0.01% to 30% by weight, preferably from 0.1% to 20% by weight, and more preferably from 0.5% to 10% by weight, relative to the total weight of the composition.

The composition according to the present invention can be prepared by mixing the ingredients (a) and (b), as essential ingredients, as well as optional ingredient(s), as explained above, in a suitable medium, such as water or oil(s).

### [Cosmetic Process]

The present invention also relates to: a cosmetic process for a keratin substrate, such as skin, comprising applying to the keratin substrate the composition according to the present invention.

The composition according to the present invention may preferably be used as a cosmetic composition. The cosmetic composition may be a sun care composition for protecting keratinous substances, such as skin, from UV rays.

The cosmetic process here means a non-therapeutic cosmetic method for caring for and/or making up the surface of a keratin substrate such as skin.

Therefore, the present invention relates to a cosmetic process for protecting keratinous substances from UV radiation, comprising, at least one step of applying the composition according to the present invention to the keratinous substance, such as skin.

### [Use]

The present invention also relates to a use of (b) at least one hollow silica sphere particle to enhance the UV absorbance of the non-pulverulent composition comprising (a) at least one lipophilic organic UV filter.

The above explanations regarding the (a) at least one lipophilic organic UV filter, (b) at least one hollow silica sphere particle, the non-pulverulent composition as well as the other optional ingredients which can be included in the non-pulverulent composition can apply to those in the above use according to the present invention.

The inventors have surprisingly discovered that the (b) hollow silica sphere particles can increase a UV absorbance of non-pulverulent compositions comprising the (a) lipophilic organic UV filter.

Therefore, the use of the (b) hollow silica sphere particles can provide a sufficient UV protecting property with the non-pulverulent compositions comprising the (a) lipophilic organic UV filter. This fact can lead to a reduction of an amount of UV filters, such as lipophilic organic UV filters, which may cause negative impacts on cosmetic compositions, for example, oily or greasy texture, in cosmetic non-pulverulent compositions. Accordingly, the use according to the present invention can provide good UV protecting effects as well as good texture in consideration of a reduced amount of UV filters, in particular lipophilic organic UV filters, in sun care non-pulverulent compositions.

Therefore, the present invention also relates to a use of (b) at least one hollow silica sphere particle as an SPF booster in non-pulverulent cosmetic composition comprising (a) at least one lipophilic organic UV filter.

### EXAMPLES

The present invention will be described in more detail by way of examples which however should not be construed as limiting the scope of the present invention.

### Example 1 and Comparative Examples 1 and 2

### [Compositions]

Each of the W/O emulsion compositions according to Example 1 (Ex. 1) and Comparative Examples 1 and 2 (Comp. Ex. 1 and Comp. Ex. 2) was prepared by mixing ingredients listed in the following Table 1 so as to be homogeneous. The formulations are also shown in the following Table 1. Among the ingredients, the hollow silica sphere particle was obtained from the company JGC Catalysts and Chemicals (product name: BA4). This hollow silica sphere particle was non-porous and had an average primary particle size of 4μm, BET specific surface area of 3 m²/g, a void ratio according to the inside cavity of 36%, an oil absorption capacity of 50 mL/100g measured with JIS-K5101 using boiled linseed oil, a specific gravity of 1.4 g/cm³, and a refractive index of 1.2.

The numerical values for the amounts of the ingredients are all based on “% by weight” as active raw materials.

### [Evaluation]

### (UV Absorbance)

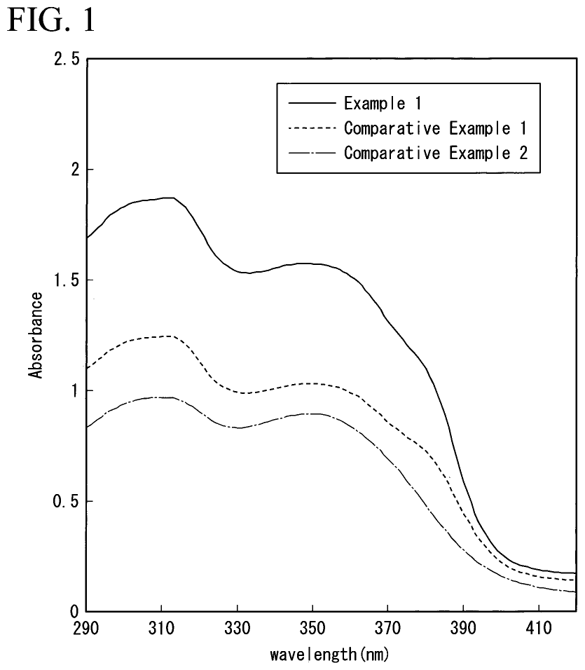

Each of the compositions was transferred onto a plate (Helio plate HD 6, PMMA, roughness: 6tim) with an adjustable pipette in an amount of 20 mg/cm² of the plate and then uniformly spread by the fingers. The coated plate was air dried for 15 minutes at room temperature. The obtained sample plate was placed into a Labsphere Ultraviolet Transmittance Analyzer (Model UV-2000 from Solar Light Company, Philadelphia, Pa.). Irradiation of UV rays took place at 12 points on the sample plate. The UV absorbance by the sample plate in the wavelength of from 250 to 420 nm was recorded. The results are shown in Table 1 and FIG. 1.

TABLE 1

| | | Comp. | Comp. |
|---|---|---|---|
| Ingredients | Ex.1 | Ex.1 | Ex.2 |
| Octocrylene | 5 | 5 | 5 |
| Drometrizole Trisiloxane | 1 | 1 | 1 |
| Butyl Methoxydibenzoylmethane | 3 | 3 | 3 |
| Ethylhexyl Triazone | 1.5 | 1.5 | 1.5 |
| Diethylamino Hydroxybenzoyl | 1 | 1 | 1 |
| Hexyl Benzoate | | | |
| Homosalate | 7 | 7 | 7 |
| Caprylyl Glycol | 0.5 | 0.5 | 0.5 |
| Poly C10-30 Alkyl Acrylate | 0.2 | 0.2 | 0.2 |
| Polyglyceryl-6 Polyricinoleate | 0.5 | 0.5 | 0.5 |
| Octyldodecanol (and) | 1.5 | 1.5 | 1.5 |
| Octyldodecyl Xyloside | | | |
| PEG-10 Dimethicone | 0.5 | 0.5 | 0.5 |
| Dicaprylyl Ether | 5 | 5 | 5 |
| Diisopropyl Sebacate | 5 | 5 | 5 |
| Dimethicone | 15.3 | 25.3 | 15.3 |
| Disteardimonium Hectorite | 1 | 1 | 1 |
| (and) Propylene Carbonate | | | |
| Titanium Dioxide (and) | 6 | 6 | 6 |
| Aluminum Hydroxide (and) | | | |
| Dimethicone (and) Hydrogen | | | |
| Dimethicone | | | |
| Dimethicone (and) Dimethicone/ | 2 | 2 | 2 |
| Vinyl Dimethicone | | | |
| Crosspolymer | | | |
| Propylene Glycol | 3 | 3 | 3 |
| Trisodium Ethylenediamine | 0.1 | 0.1 | 0.1 |
| Disuccinate | | | |
| Hollow Silica Sphere Particle | 10 | — | — |
| Calcium Aluminum Borosilicate | — | — | 10 |
| Phenoxyethanol | 0.7 | 0.7 | 0.7 |
| Ethanol | 4.9 | 4.9 | 4.9 |
| Tocopherol | 0.1 | 0.1 | 0.1 |
| Water | q.s. 100 | q.s. 100 | q.s. 100 |
| Evaluation | | | |
| Absorbance at 311 nm | 1.87 | 1.25 | 0.97 |
| Absorbance at 347 nm | 1.58 | 1.03 | 0.89 |

It is clear from Table 1 and FIG. 1 that the composition according to Example 1, which includes a combination of at least one lipophilic organic UV filter and at least one hollow silica sphere particle, has a higher UV absorbance than the composition according to Comparative Example 1, which does not include the combination, and Comparative Example 2, which includes calcium aluminum borosilicate instead of the hollow silica sphere particle in the composition of Example 1, especially from 290 nm to 380 nm. It can be recognized that the higher UV absorbance is provided by the use of the hollow silica sphere particle together with lipophilic organic UV filter(s). This fact is surprising because it is well-known that hollow silica sphere particles themselves do not exhibit particular UV absorbance properties. Also, it is surprising that the composition according to Comparative Example 2, which includes calcium aluminum borosilicate instead of the hollow silica sphere particle in the composition of Example 1, showed even lower UV absorbance than the composition according to Comparative Example 1, which does not include any comparable inorganic fillers. Accordingly, it can be said that these results showed very remarkable property of hollow silica sphere particles which can enhance UV absorbance due to lipophilic organic UV filter(s).

Therefore, it can be concluded that the particle according to the present invention can be very preferable as an SPF booster ingredient to filter out UV rays in sun care non-pulverulent cosmetics.

### Example 2

A solution composition according to Example 2 (Ex. 2) was prepared by mixing ingredients listed in the following Table 2 so as to be homogeneous. The formulation is also shown in the following Table 2. Among the ingredients, the hollow silica sphere particle was the same as that used in Example 1 above.

TABLE 2

| Ingredients | Ex. 2 |
|---|---|
| Water | q.s. 100 |
| Phenoxyethanol | 0.5 |
| Ethanol | 4.9 |
| Glycerin | 3.5 |
| Phenylbenzimidazole Sulfonic Acid | 1 |
| Tromethamine | 0.5 |
| Terephthalylidene Dicamphor | 6 |
| Sulfonic Acid | |
| Triethanolamine | 1.1 |
| Isopropyl Myristate | 0.9 |
| Diisopropyl Sebacate | 0.1 |
| Polyglyceryl-4 Caprate | 0.9 |
| Polyglyceryl-2 Oleate | 0.3 |
| Butyl Methoxydibenzoylmethane | 4 |
| Octocrylene | 7 |
| Homosalate | 8 |
| Ethylhexyl Salicylate | 4 |
| Hollow Silica Sphere Particle | 4 |

1. A non-pulverulent cosmetic composition, comprising:
(a) at least one lipophilic organic UV filter, and
(b) at least one hollow silica sphere particle.

2. The composition according to claim 1, wherein the (b) hollow silica sphere particle is non-porous.

3. The composition according to claim 1, wherein the (b) hollow silica sphere particle has an average primary particle size of 0.5 μm or more and of 100 μm or less.

4. The composition according to claim 1, wherein the (b) hollow silica sphere particle has a specific surface area determined by BET method of 50 m²/g or less and of 0.1 m²/g or more.

5. The composition according to claim 1, the (b) hollow silica sphere particle has a void ratio of 70% by volume or less and of 10% by volume or more.

6. The composition according to claim 1, wherein the (b) hollow silica sphere particle has an oil absorption capacity of 200 mL/100g or less and of 10 mL/100g or more.

7. The composition according to claim 1, wherein the (b) hollow silica sphere particle has a specific gravity of 1.1 g/cm³ or more and of 1.7 g/cm³ or less.

8. The composition according to claim 1, wherein the (a) lipophilic organic UV filter comprises a combination of at least one lipophilic organic UV-A filter and at least one lipophilic organic UV-B filter.

9. The composition according to claim 1, wherein the (a) lipophilic organic UV filter is selected from minobenzophenone compounds, dibenzoylmethane compounds, triazine compounds, salicylic compounds, β,β-diphenylacrylate compounds, and benzotriazole compounds, and mixtures thereof.

10. The composition according to claim 1, which is in the form of a W/O emulsion or a solution.

11. The composition according to claim 1, wherein the amount of the (a) lipophilic organic UV filter is 1% by weight or more and is 50% by weight or less relative to the total weight of the composition.

12. The composition according to claim 1, wherein the amount of the (b) hollow silica sphere particle is 0.5% by weight or more and is 40% by weight or less relative to the total weight of the composition.

13. The composition according to claim 1, which includes inorganic fillers other than hollow silica particles in an amount of 10% by weight or less.

14. A cosmetic process for a keratin substrate, comprising applying to the keratin substrate the composition according to claim 1.

15. A process comprising: adding (b) at least one hollow silica sphere particle to a non-pulverulent composition comprising (a) at least one lipophilic organic UV filter to enhance UV absorbance thereof.
